(19) United States
(12) Patent Application Publication
(10) Pub. No.: US 2026/0207234 A1
(43) Pub. Date: Jul. 23, 2026

(54) IMPLANT DEVICE FOR A TIBIAL PLATEAU LEVELING OSTEOTOMY

(71) Applicant: The TPLO Company LLC, Carson City, NV (US)

(72) Inventor: Claude Luc Gendreau, Highland, IN (US)

(73) Assignee: The TPLO Company LLC, Carson City, NV (US)

(21) Appl. No.: 19/420,983

(22) Filed: Dec. 16, 2025

Related U.S. Application Data
(63) Continuation-in-part of application No. 19/029,561, filed on Jan. 17, 2025, now Pat. No. 12,502,211.

(51) Int. Cl. A61B 17/80; A61F 2/08; A61F 2/30

(52) U.S. Cl. CPC A61B 17/8061; A61F 2/08; A61F 2002/307; A61F 2230/0039

(57) ABSTRACT

An implant device that includes: an epiphyseal and a metaphyseal tibial plate section comprising at least two screw holes and a bone-contacting surface, each screw hole for securing the respective plate section to a respective epiphyseal or metaphyseal tibial bone fragment; and a ligament that joins the tibial plate sections and is configured to span a curvilinear tibial cut having a concave shape with a distally inward orientation between the bone fragments. Each of the tibial plate sections comprises a tibial plate length and width, and each length is at least two times longer than the respective width. The width of each of the plate sections may be substantially uniform along the length of its respective section. In addition, each of the tibial plate sections comprises a cut-adjacent curvilinear surface that has a shape (e.g., a concave shape) that substantially matches the shape and inward orientation of the curvilinear cut.

### CROSS-REFERENCE TO RELATED APPLICATIONS

This application claims the benefit of priority under 35 U.S.C. § 120 as a continuation-in-part application of U.S. Non-Provisional patent application Ser. No. 19/029,561, filed Jan. 17, 2025 and entitled “Implant Device for a Tibial Plateau Leveling Osteotomy”, the entire contents of which are hereby incorporated by reference within this disclosure and application.

### FIELD OF THE DISCLOSURE

The present disclosure relates to a surgical implant device used as part of a tibial plateau leveling osteotomy (TPLO) procedure and, more specifically, a compact implant device for an animal (e.g., a canine) undergoing a tibial plateau leveling osteotomy (TPLO) procedure that reduces incision size, procedure duration, the risk of infection, and provides a more secure fixation.

### BACKGROUND

As is well known in the field, the cranial cruciate ligament (CCL) in many animals can partially rupture, fully rupture, or otherwise degenerate over time. The CCL provides stability to the animal's stifle joint by preventing backward movement of the femur relative to the tibia. Various breeds of dogs and other animals are born with or otherwise develop an exaggerated backward slope of the upper surface (plateau) of their tibia bones. This slope contributes to stress on the CCL ligament, often leading to its rupture or degeneration. Once the CCL ligament is compromised, the animal experiences pain and arthritis can quickly develop in the stifle joint.

Various TPLO implant devices are known in the field to stabilize the bone fragments in their new position following an osteotomy. During a typical TPLO procedure, a veterinary surgeon will make a curvilinear cut within the tibia, relatively close to the tibial plateau and stifle joint, separating metaphyseal and epiphyseal sections of the tibial bone. After the curvilinear cut to the tibia is completed, the surgeon then rotates the tibial plateau of the epiphyseal section relative to the metaphyseal section of the tibia to an optimal angle to reduce stress on the CCL ligament in the stifle joint. An implant device is then used to secure the new orientation of the epiphyseal section of the tibia relative to the metaphyseal section of the tibia with a plate attached to each bone section. The implant device is left in the animal after the TPLO procedure until the osteotomy is healed or indefinitely if there is no rejection of the implant and no infection.

The conventional implant devices employed in TPLO procedures have several disadvantages and problems. Most such implant devices employ a plate with an elongated appendage or protrusion affixed to the metaphyseal (distal) section of the tibia, ostensibly to ensure that the plate is securely affixed to the metaphyseal section of the tibia. However, the additional length of the appendage or protrusion of these implant devices necessitates a long surgical exposure, which can extend recovery times and lead to an increased likelihood of infection and implant rejection. Further, these relatively bulky conventional implant devices can result in lengthier TPLO procedures, which increase infection risk. In addition, conventional popular TPLO implant devices are asymmetric and configured only for use with the right or left leg of the animal.

Accordingly, there is a need for a more compact implant device design for TPLO procedures that reduces incision size, the duration of the procedure, and the risk of post-surgery infections and other complications. Such a device should further provide a more uniform securement of the epiphyseal and metaphyseal fragments separated by the curvilinear cut between the two tibial sections during and after recovery.

### SUMMARY

According to an aspect of the disclosure, an implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to the epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament that joins the two tibial plate sections and is configured to span a curvilinear tibial cut, wherein the curvilinear cut separates the epiphyseal and metaphyseal tibial bone fragments and has a concave shape with a distally inward orientation toward the metaphyseal tibial bone fragment. Each of the metaphyseal and epiphyseal tibial plate sections and the ligament comprises an implant grade alloy. Further, the epiphyseal tibial plate section comprises an epiphyseal tibial plate length and width, the metaphyseal tibial plate comprises a metaphyseal tibial plate length and width, and each tibial plate length is at least two times longer than the respective tibial plate width. In addition, each of the tibial plate sections comprises a cut-adjacent curvilinear surface that is substantially normal to its bone-contacting surface. Further, each of the cut-adjacent curvilinear surfaces has a concave shape that substantially matches the concave shape and inward orientation of the curvilinear cut.

According to an aspect of the disclosure, an implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate configured to accept a bone screw to secure the plate section to a metaphyseal bone fragment; and a ligament that joins the two tibial plate sections and is configured to span a curvilinear tibial cut, wherein the curvilinear cut separates the epiphyseal and metaphyseal tibial fragments and has a shape with a distally inward orientation toward the metaphyseal tibial bone fragment. Each of the epiphyseal and metaphyseal tibial plate sections and the ligament comprises an implant grade alloy. Further, the epiphyseal tibial plate section comprises an epiphyseal tibial plate length and width, the metaphyseal tibial plate section comprises a metaphyseal tibial plate length and width, and each tibial plate section length is at least two times longer than the respective tibial plate width. In addition, the epiphyseal tibial plate section length is shorter than the metaphyseal tibial plate section length. Each of the tibial plate sections comprises a cut-adjacent curvilinear surface that is substantially normal to its bone-contacting surface. Further, each of the cut-adjacent curvilinear surfaces has a shape that substantially matches the shape and inward orientation of the curvilinear cut.

According to an aspect of the disclosure, an implant device for a tibial plateau leveling osteotomy is provided that consists essentially of: an epiphyseal tibial plate section comprising at least two screw holes and a bone-contacting surface, each screw hole of the epiphyseal tibial plate section configured to accept a bone screw to secure the epiphyseal plate section to an epiphyseal tibial bone fragment; a metaphyseal tibial plate section comprising at least two screw holes and a bone-contacting surface, each screw hole of the metaphyseal tibial plate section configured to accept a bone screw to secure the metaphyseal plate section to a metaphyseal tibial bone fragment; and a ligament that joins the tibial plate sections and is configured to span a curvilinear tibial cut, wherein the curvilinear cut separates the epiphyseal and metaphyseal tibial fragments and has a shape with a distally inward orientation toward the metaphyseal tibial bone fragment. Each of the epiphyseal and metaphyseal tibial plate sections and the ligament comprises an implant grade alloy. Further, the epiphyseal tibial plate section comprises an epiphyseal tibial plate length and width, the metaphyseal tibial plate section comprises a metaphyseal tibial plate length and width, and each tibial plate section length is at least two times longer than the respective tibial plate width. In addition, at least one screw hole of the epiphyseal tibial plate section is devoid of threading and comprises an unbeveled region, the unbeveled region comprising 90° or less of the total circumference of the at least one screw hole. Each of the tibial plate sections comprises a cut-adjacent curvilinear surface that is substantially normal to its bone-contacting surface. Further, each of the cut-adjacent curvilinear surfaces has a shape that substantially matches the shape and inward orientation of the curvilinear cut.

Additional features and advantages will be set forth in the detailed description which follows, and in part will be readily apparent to those skilled in the art from that description or recognized by practicing the embodiments as described herein, including the detailed description which follows, the claims, as well as the appended drawings.

### BRIEF DESCRIPTION OF THE DRAWINGS

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments, wherein:

FIG. 1A is a perspective view of an implant device with epiphyseal and metaphyseal tibial plate sections and a ligament, according to an embodiment of the disclosure;

FIG. 1B is a top plan view of the implant device of FIG. 1A;

FIG. 1C is a bottom plan view of the implant device of FIG. 1A;

FIG. 1D is an end-on view of the implant device of FIG. 1A along an axis parallel to the length of its epiphyseal and metaphyseal tibial plate sections;

FIG. 2A is a perspective front view of an implant assembly comprising the implant device of FIG. 1A, as secured to epiphyseal and metaphyseal tibial bone fragments across a curvilinear tibial cut, according to an embodiment of the disclosure;

FIG. 2B is a 90° counterclockwise-rotated perspective view of the implant assembly of FIG. 2A;

FIG. 2C is a ~10° clockwise-rotated perspective view of the implant assembly of FIG. 2A;

FIG. 3 is a top plan view of nine (9) implant devices of varying sizes and bone screw hole configurations for animals of various sizes, according to embodiments of the disclosure;

FIG. 4 is a top plan view of an osteotomy implant device with epiphyseal and metaphyseal tibial plate sections and a ligament, according to another embodiment of the disclosure; and

FIG. 5 is a perspective view of an implant device with epiphyseal and metaphyseal tibial plate sections and a ligament, according to an embodiment of the disclosure.

### DETAILED DESCRIPTION

In the following detailed description, for purposes of explanation and not limitation, example embodiments disclosing specific details are set forth to provide a thorough understanding of various principles of the present disclosure. However, it will be apparent to one having ordinary skill in the art, having had the benefit of the present disclosure, that the present disclosure may be practiced in other embodiments that depart from the specific details disclosed herein. Moreover, descriptions of well-known devices, methods and materials may be omitted so as not to obscure the description of various principles of the present disclosure. Finally, wherever applicable, like reference numerals refer to like elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example “up,” “down,” “right,” “left,” “front,” “back,” “top,” “bottom”—are made only with reference to the figures as drawn and are not intended to imply absolute orientation.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

As used herein, the singular forms “a,” “an” and “the” include plural referents unless the context clearly dictates otherwise. Thus, for example, reference to a “component” includes aspects having two or more such components, unless the context clearly indicates otherwise.

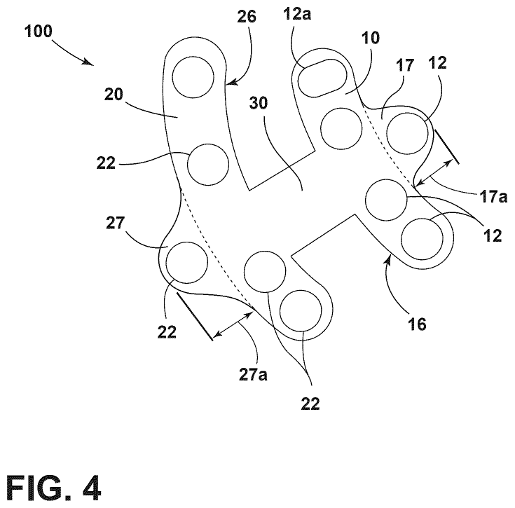

As also used herein, the term “substantially uniform”, as used in connection with the width of one or more plate sections of the implant device of the disclosure, means that the width does not vary substantially along the length of the plate section. More specifically, a “substantially uniform” width of the plate section cannot vary by more than 100% of the width of the plate section at any one location along the length of the plate section. Consequently, one or more plate sections of the implant device of the disclosure may, in some implementations, possess one or more short, support protrusions or appendages (see FIG. 4); however, these short, support protrusions or appendages are limited in width to no more than 100% of the width of the plate section.

As used herein, the term “substantially matches” is used to describe the cut-adjacent surfaces and/or the shapes of the plate sections of the implant devices of the disclosure as to their similarity in both shape and alignment to the shape and alignment of the curvilinear cut to a tibial bone that results from a TPLO procedure. Unless otherwise noted, the cut-adjacent surfaces and/or the shapes of the plate sections in question substantially match when a low normalized root mean squared error (NRMSE) value (i.e., NRMSE<0.5) is calculated from a comparison between the curve of the curvilinear cut and the curve of the cut-adjacent surface, as would be understood by those skilled in the field of the disclosure.

Generally, the present disclosure is directed to compact implant device designs for TPLO procedures that reduce incision size, procedure duration, and the risk of post-surgery infections and other complications. According to a typical TPLO procedure, a veterinary surgeon will make a curvilinear cut within the tibia, relatively close to the tibial plateau and stifle joint, and separate metaphyseal and epiphyseal sections of the tibial bone. The implant device designs of the disclosure maintain an effective, uniform securement of the curvilinear cut (i.e., as formed by the TPLO procedure) between the tibial sections during and after recovery of the animal (e.g., a canine).

Tibial plateau osteotomy implant devices of the disclosure comprise, or consist essentially of, an epiphyseal tibial plate section, a metaphyseal tibial plate section, and a ligament configured to join the tibial plate sections, with each of the plate sections and ligaments comprising an implant grade alloy. Each of the plate sections includes at least two screw holes, each screw hole configured to accept a bone screw to secure the respective plate section to the respective epiphyseal or metaphyseal tibial bone fragment. Further, the length of each of the plate sections is at least two times the width of each of the plate sections. In some aspects, the width of each of the plate sections can be substantially uniform along the length of the plate section. In some aspects of these devices, each of the plate sections comprises a cut-adjacent curvilinear surface with a plate radius within ±25% of the curvilinear cut radius of the curvilinear cut between the tibial bone fragments. In some aspects of these devices, the epiphyseal tibial plate length is shorter than the metaphyseal tibial plate length.

The implant devices of the disclosure offer several advantages over conventional implant devices employed in TPLO procedures. The implant device configurations of the disclosure are compact, which facilitates relatively shorter incision sizes, shorter operation durations, lower infection risk, and lower risk of implant device rejection. Further, the implant device configurations of the disclosure are readily adaptable for TPLO procedures employed on animals of varying sizes (e.g., different dog breeds). In addition, the implant devices of the disclosure are expected to distribute stress more uniformly across the curvilinear cut to the tibia from the TPLO procedure. Moreover, embodiments of the implant device configurations of the disclosure have symmetry such that they can be configured for use with both right and left legs of animals.

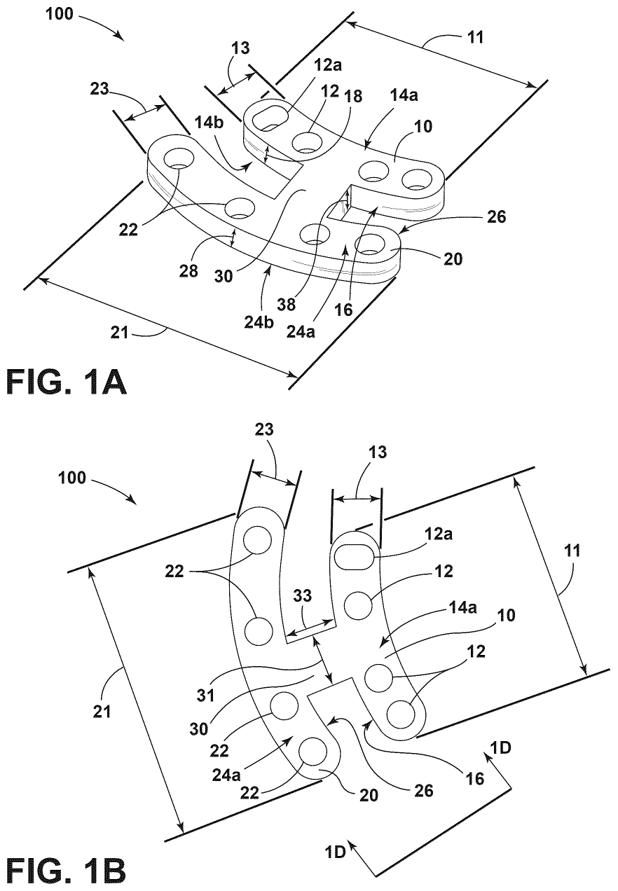

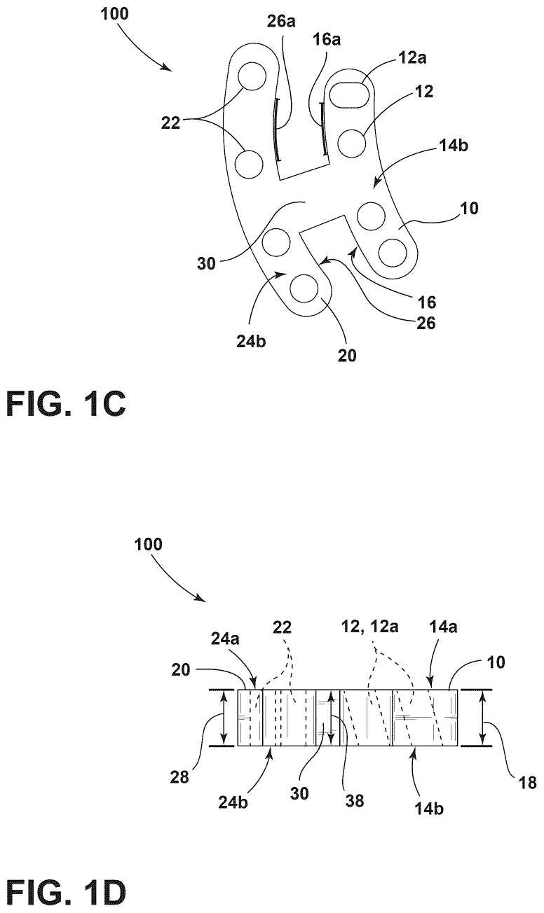

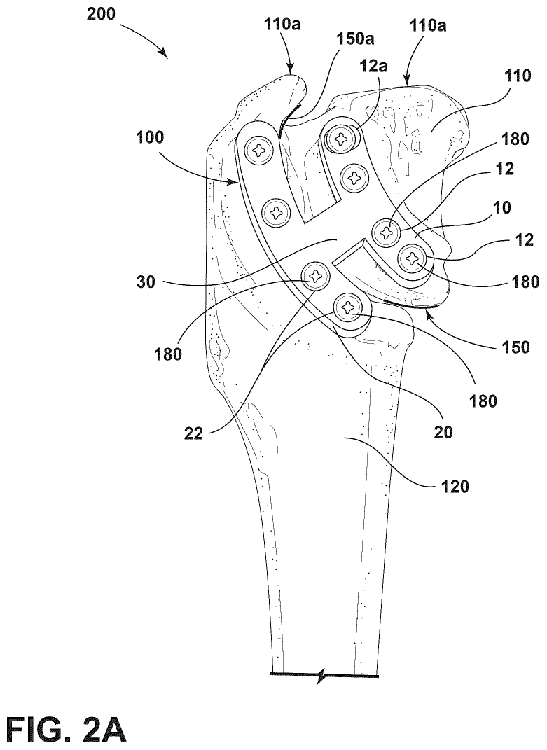

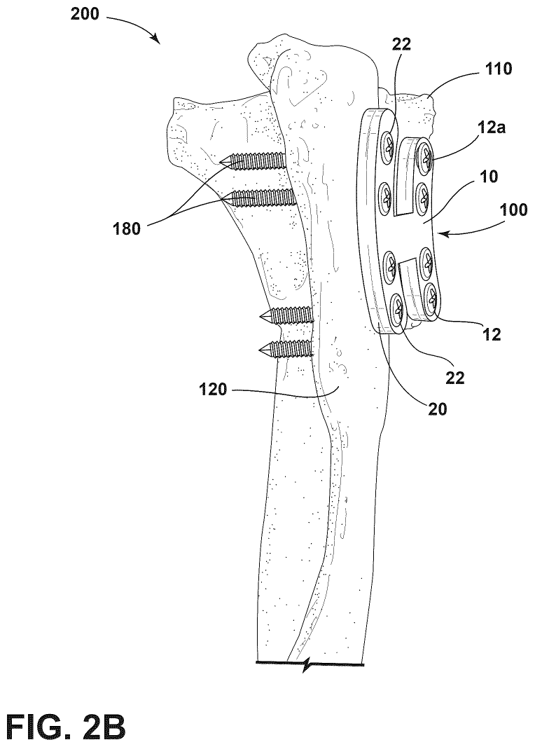

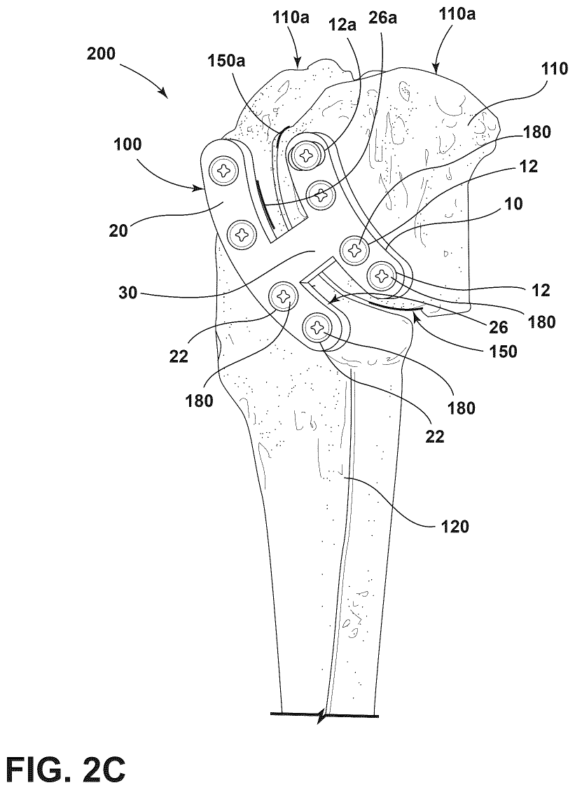

Referring now to FIGS. 1A-1D, an implant device 100 for a TPLO procedure (see FIGS. 2A-2C, described further below) is depicted. The implant device 100 shown in FIGS. 1A-1D includes an epiphyseal tibial plate section 10 with at least two screw holes 12, an exterior surface 14a, and a bone-contacting surface 14b. Unless otherwise noted, each screw hole 12 of the epiphyseal tibial plate section 10 has a round shape and is configured to accept bone screws 180 to secure the plate section 10 to the epiphyseal tibial bone fragment 110 (see FIGS. 2A-2C). In some implementations of the implant device 100, one screw hole 12a (e.g., the proximal-most screw hole) of the epiphyseal tibial plate section 10 can be elongated in a direction perpendicular to a curvilinear cut 150 of the tibial bone, as shown in exemplary form in FIGS. 1A-1D. In general, the epiphyseal tibial plate section 10 is configured to attach to an epiphyseal tibial bone fragment 110 that results from the TPLO procedure and, particularly, a curvilinear cut 150 to a tibial bone. The epiphyseal tibial plate section 10 also functions to distribute stress (e.g., from movement of the tibia and stifle joint associated with movement by the animal) uniformly across a curvilinear tibial cut 150. In embodiments, the epiphyseal tibial plate section 10 has a curvilinear shape (see FIG. 1A), e.g., to substantially match the shape of the curvilinear tibial cut 150. According to some embodiments, the epiphyseal tibial plate section 10 of the implant device 100 shown in FIGS. 1A-1D can comprise any of various shapes with a length 11 at least two times its width 13, including an elliptical, curved, curvilinear, or kidney bean-like shape.

Referring again to FIGS. 1A-1D, the TPLO implant device 100 further includes a metaphyseal tibial plate section 20 with at least two screw holes 22, an exterior surface 24a, and a bone-contacting surface 24b. Each screw hole 22 of the metaphyseal tibial plate section 20 is configured to accept a bone screw 180 to secure the plate section 20 to a metaphyseal tibial bone fragment 120 (see FIGS. 2A-2C, as described further below). The metaphyseal tibial plate section 20 is generally configured to secure a metaphyseal tibial bone fragment 120 defined from the TPLO procedure and, particularly, the curvilinear cut 150 to the tibial bone. The metaphyseal tibial plate section 20 also serves to distribute stress uniformly across the curvilinear tibial cut 150. In embodiments, the metaphyseal tibial plate section 20 has a curvilinear shape (see FIG. 1A), e.g., to substantially match the shape of the curvilinear tibial cut 150. According to some embodiments, the metaphyseal tibial plate section 20 can comprise any of various shapes with a length 21 at least two times its width 23, including an elliptical, curved, curvilinear, or kidney bean-like shape. In some implementations, the shapes of the epiphyseal and metaphyseal tibial plate sections 10, 20 substantially match and match the shape of the curvilinear tibial cut 150. In other implementations, the shapes of the plate sections 10, 20 can differ from one another.

In some embodiments of the implant device 100, as depicted in exemplary form in FIGS. 1A-1D, the metaphyseal tibial plate section 20 comprises an elliptical, curved, curvilinear, or kidney bean-like shape with no appendage or protrusion that extends in the proximal direction away from the curvilinear cut 150. Further, in these embodiments, the tibial plate sections 10, 20 can have a high aspect ratio of at least 2:1 to ensure that the device is compact in size. As noted earlier, the compact size of the implant device 100 can facilitate relatively straightforward TPLO procedures with small incision sizes, short durations, and lower infection risk. In addition, the compact size of the implant device 100 employing tibial plate sections 10, 20 with high aspect ratios (at least 2:1) and no appendages or protrusions emanating from the metaphyseal tibial plate section 20 also ensures that stresses can be uniformly distributed across the curvilinear cut 150, thus ensuring that the device 100 offers a stable securement of the epiphyseal bone fragment following the osteotomy.

Still referring to FIGS. 1A-1D, the TPLO implant device 100 further includes a ligament 30 that joins the epiphyseal and metaphyseal tibial plate sections 10, 20. In embodiments, the ligament 30 is integral with the epiphyseal and metaphyseal tibial plate sections 10, 20 such that the implant device 100 can be fabricated from a single plate. In other embodiments (not shown), the ligament 30 is joined or otherwise secured to both of the tibial plate sections 10, 20 by any appropriate method as understood by those skilled in the field of the disclosure appropriate for the material(s) (e.g., an implant grade metal alloy) of each of the plate sections 10, 20 and ligament 30 (e.g., welding, fastening, brazing, riveting, hot-pressing, etc.).

Referring again to FIGS. 1A-1D, the ligament 30 of the TPLO implant device 100 is configured to span the curvilinear tibial cut 150, in addition to joining the epiphyseal and metaphyseal tibial plate sections 10, 20. The ligament 30 can be defined by a ligament length 31 defined along an axis substantially parallel to an axis defined by each of the epiphyseal and metaphyseal tibial plate section lengths 11, 21. The ligament 30 can also be defined by a ligament width 33, the width 33 dimension defined as being substantially normal to the ligament length 31. In some embodiments, the ligament length 31 is at least about 35% (e.g., a width 33 of <10.4 mm and plate section lengths 11, 21=16 mm), 40%, 50%, 60%, 70%, 80%, 90%, or 95% shorter than each of the tibial plate section lengths 11, 21, e.g., to ensure that the surgeon is able to observe the curvilinear cut 150 beside and/or through the ligament 30, and properly orient the TPLO implant device 100 during the TPLO procedure. Further, as depicted in exemplary form in FIGS. 1A-1D, the ligament 30 can comprise a single connection between the tibial plate sections 10, 20. In some implementations (not shown), the ligament 30 can comprise multiple bands, joints, ligaments, or other elements situated between the tibial plate sections 10, 20 to join them and facilitate visibility to the curvilinear cut 150 beneath the TPLO implant device 100. In such implementations, the multiple bands, joints, ligaments, or other elements collectively define the ligament length 31 and width 33.

Referring again to FIGS. 1A-1D, each of the metaphyseal and epiphyseal tibial plate sections 10, 20 and the ligament 30 can be fabricated from an implant grade alloy, as understood by those skilled in the field of the disclosure. Suitable implant grade alloys include stainless steel alloys (e.g., SS 316, SS 316LVM, SS 304, and 17-4 PH) titanium alloys (e.g., Ti-6Al-4V, Ti-6Al-7Nb, Grade 2, and Grade 4). Each of the tibial plate sections 10, 20 and ligament 30 can be fabricated from the same implant grade alloy. In addition, each of the bone screws 180 employed in connection with a TPLO procedure to install an implant device 100 of the disclosure is fabricated from the same implant grade alloy as used for the plate sections 10, 20 and the ligament 30 (see, e.g., FIGS. 2A-2C). Further, various surface finishes and roughness levels of the epiphyseal tibial plate section 10, metaphyseal tibial plate section 20 and ligament 30 are acceptable, as deemed by those skilled in the field of the disclosure to be suitable for the application and manufacturing of the TPLO implant device 100. In an embodiment of the implant device 100, each of the epiphyseal tibial plate section 10, metaphyseal tibial plate section 20 and ligament 30 are manufactured such that their surfaces (e.g., bone-contacting surfaces 14b, 24b) can have a matte finish (e.g., with a surface roughness from 20 to 60 μm (Rₐ)). According to an embodiment of the implant device 100 depicted in exemplary form in FIGS. 1A-1D, one or both of the bone-contacting surfaces 14b, 24b are manufactured with a dimpled, roughened or textured surface (e.g., with a surface roughness of greater than 60 μm (Rₐ)). In some embodiments, the epiphyseal tibial plate section 10 and/or the ligament 30 can be angled laterally up to +/−10° or +/−5° relative to the bone-contacting surface 24b of the metaphyseal tibial plate section 20 to accommodate slight misalignment between the bone fragments 110, 120 (not shown).

Still referring to the TPLO implant device 100 of FIGS. 1A-1D, the epiphyseal tibial plate section 10 can be defined by an epiphyseal tibial plate section length 11 and width 13, and the metaphyseal tibial plate section 20 can be defined by a metaphyseal tibial plate section length 21 and width 23. Further, according to some implementations of the implant device 100, the width 13, 23 of each of the tibial plate sections 10, 20 is substantially uniform along the length 11, 21 of each of the respective tibial plate sections 10, 20. Consequently, in some embodiments, one or both of the plate sections 10, 20 can have one or more short, support protrusions 17, 27 (see FIG. 4, described further below); however, these short, support protrusions 17, 27 are limited in width 17a, 27a to no more than 100% of the width 13, 23 of the plate sections 10, 20. In some embodiments, the width 13, 23 of each of the tibial plate sections 10, 20 is both uniform and varies by no more than +10%, 5%, 1%, 0.1%, 0.01%, or even less, as only limited by machining capability and the materials selected for the tibial plate sections 10, 20. In some implementations of the implant devices 100, the width 13, 23 of each of the tibial plate sections can vary by more than 100% at one or more points or regions along the length 11, 21 of the tibial plate sections 10, 20 (not shown in FIGS. 1A-1D). Further, according to some embodiments of the implant device 100, each tibial plate section length 11, 21 is at least two times, three times, or four times longer than the respective tibial plate section width 13, 23.

According to embodiments of the TPLO implant device 100 shown in exemplary form in FIGS. 1A-1D, the aspect ratio of each of the tibial plate sections 10, 20 (i.e., ratio of length 11, 21 to width 13, 23, respectively) is at least 2:1, 2.5:1, 3:1, 3.5:1, 4:1, 5:1, and all aspect ratios between and greater than the foregoing aspect ratios. With regard to these embodiments, an implant device 100 with tibial plate sections 10, 20 having high aspect ratios of at least 2:1 ensures that the device is compact in size. Further, as noted earlier, the compact size of the implant device 100 can facilitate straightforward TPLO procedures with small incision sizes, short durations, and low infection risk. In addition, the compact size of the implant device 100 employing tibial plate sections 10, 20 with high aspect ratios (at least 2:1) also ensures that stresses can be uniformly distributed across the curvilinear cut 150, thus ensuring that the device 100 offers a stable securement of the proximal tibia following the osteotomy of the TPLO procedure.

Referring again to the TPLO implant device 100 depicted in FIGS. 1A-1D, according to some embodiments, the epiphyseal tibial plate section 10 can be configured such that its plate section length 11 is from 8 mm to 36 mm, and its width 13 is from 4 mm to 10 mm. In some implementations, the epiphyseal plate section length 11 can be 8 mm, 10 mm, 12 mm, 14 mm, 16 mm, 18 mm, 20 mm, 22 mm, 24 mm, 26 mm, 28 mm, 30 mm, 32 mm, 34 mm, 36 mm, or any length 11 between any of the foregoing length values. In some implementations, the epiphyseal plate section width 13 can be 4 mm, 5 mm, 6 mm, 7 mm, 8 mm, 9 mm, 10 mm, or any width 13 between any of the foregoing width values. For example, according to some embodiments, the length 11 of the epiphyseal tibial plate section 10 can be 2 times, 2.5 times, 3 times, 3.5 times, 4 times, 4.5 times, 5 times, or any factor between the foregoing, of its width 13. Unless otherwise noted, the plate section length 11 is defined as the longest dimension of the epiphyseal plate section 10 and the plate section width 13 is defined as the shorter dimension of the epiphyseal plate section 10 along its length 11.

Still referring to the TPLO implant device 100 depicted in FIGS. 1A-1D, according to some embodiments, the metaphyseal tibial plate section 20 can be configured such that its plate section length 21 is from 10 mm to 38 mm, and its width 23 is from 4 mm to 10 mm. In some implementations, the metaphyseal plate section length 21 can be 10 mm, 12 mm, 14 mm, 16 mm, 18 mm, 20 mm, 22 mm, 24 mm, 25 mm, 26 mm, 28 mm, 30 mm, 32 mm, 34 mm, 36 mm, 38 mm, or any length 21 between any of the foregoing length values. In some implementations, the metaphyseal plate section width 23 can be 4 mm, 5 mm, 6 mm, 7 mm, 8 mm, 9 mm, 10 mm, or any width 23 between any of the foregoing width values. For example, according to some embodiments, the length 21 of the metaphyseal tibial plate section 20 can be 2 times, 2.5 times, 3 times, 3.5 times, 4 times, 4.5 times, 5 times, or any factor between the foregoing, of its width 23. Further, according to some embodiments of the implant device 100, as shown in exemplary form in FIGS. 1A-1D, the ends of the metaphyseal tibial plate section 20 can have different lengths, with the proximal end longer than the distal end. In other embodiments, the ends of the metaphyseal tibial plate section 20 have substantially the same length. Further, unless otherwise noted, the plate section length 21 is defined as the longest dimension of the metaphyseal plate section 20 and the plate section width 23 is defined as the shorter dimension of the metaphyseal plate section 20 along its length 21.

Referring again to the TPLO implant device 100 depicted in FIGS. 1A-1D, according to some embodiments, the ligament 30 can be configured such that its length 31 is from 4 mm to 10 mm, 4 mm to 12 mm, 4 mm to 15 mm, 4 mm to 20 mm, 4 mm to 25 mm, 4 mm to 30 mm, and all ligament lengths 31 between the foregoing length ranges. Further, the ligament 30 can be configured such that its width 33 is from 4 mm to 10 mm, 4 mm to 8 mm, 4 mm to 6 mm, and all ligament widths 33 between the foregoing width ranges. As noted earlier, in some embodiments of the implant device 100, the ligament length 31 can be at least about 35% shorter than each of the tibial plate section lengths 11, 21, which, as also noted earlier, can range from 8 mm to 36 mm and 10 mm to 38 mm, respectively. For example, the ligament length 31 can be 35%, 40%, 50%, 55%, 60%, 70%, 80%, 90%, 95%, or even 100% shorter than each of the tibial plate section lengths 11, 21. These dimensional configurations of the ligament 30 can ensure that the surgeon is able to observe the curvilinear cut 150 beside and/or through the ligament 30, and properly orient the implant device 100 during the TPLO procedure.

According to implementations of the TPLO implant device 100 depicted in FIGS. 1A-1D, each of the epiphyseal tibial plate section 10, metaphyseal tibial plate section 20 and ligament 30 can be further defined by respective thicknesses 18, 28, and 38. In some embodiments, the thickness 18 of the epiphyseal tibial plate section 10 can range from 1 mm to 5 mm. For example, the thickness 18 can be 1 mm to 4 mm, 1.25 mm to 5 mm, 1.25 mm to 4 mm, 1.5 mm to 5 mm, 1.5 mm to 4 mm, and all thicknesses 18 between the foregoing thickness values. In some embodiments, the thickness 28 of the metaphyseal tibial plate section 20 can range from 1 mm to 5 mm. For example, the thickness 28 can be 1 mm to 4 mm, 1.25 mm to 5 mm, 1.25 mm to 4 mm, 1.5 mm to 5 mm, 1.5 mm to 4 mm, and all thicknesses 28 between the foregoing thickness values. Further, according to some embodiments, the thickness 38 of the ligament 30 can range from 1 mm to 5 mm. For example, the thickness 38 can be 1 mm to 4 mm, 1.25 mm to 5 mm, 1.25 mm to 4 mm, 1.5 mm to 5 mm, 1.5 mm to 4 mm, and all thicknesses 38 between the foregoing thickness values.

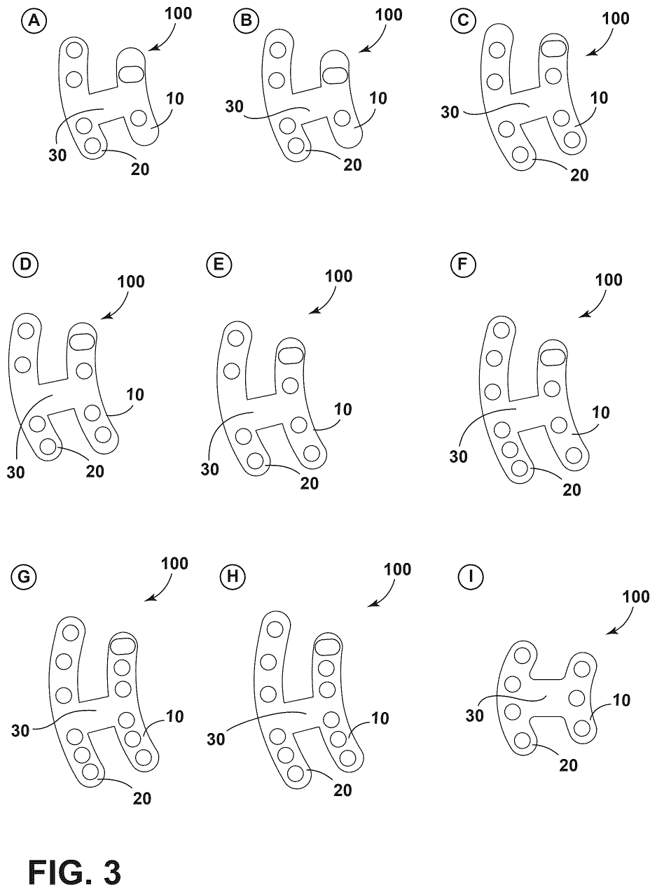

Referring again to the TPLO implant device 100 depicted in FIGS. 1A-1D, each of the epiphyseal and metaphyseal tibial plate sections 10, 20 can include at least two (2) screw holes 12, 22, respectively. In some embodiments, each of the epiphyseal and metaphyseal tibial plate sections 10, 20 can include from two (2) to ten (10), two (2) to eight (8), or two (2) to six (6) screw holes 12, 22, respectively, along with any number of screw holes 12, 22 between the foregoing ranges. For example, each of the epiphyseal and metaphyseal tibial plate sections 10, 20 can include 2, 3, 4, 5, 6, 7, 8, 9, or even 10 screw holes 12, 22, respectively. Further, according to some implementations of the implant device 100, one of the screw holes 12 of the epiphyseal tibial plate section 10 can be a single elongated screw hole 12a (e.g., the proximal-most screw hole) that is elongated in a direction perpendicular to a curvilinear cut 150 of the tibial bone to receive a round screw 180, as shown in exemplary form in FIGS. 1A-1D. According to embodiments, each of the screw holes 12, 22 comprises a round shape suitable to receive a screw 180, which also comprises a round shaped head. In some embodiments, each of the plate sections 10, 20 has the same number of screw holes 12, 12a, and 22, respectively. In other embodiments, particularly those in which the metaphyseal tibial plate section length 21 is longer than the epiphyseal plate section length 11, the number of screw holes 22 in the metaphyseal tibial plate section 20 is greater than the number of screw holes 12 in the epiphyseal tibial plate section 10. In general, the spacing and arrangement of the screw holes 12, 12a, and 22, respectively, in the plate sections 10, 20 can be equal or differ, as determined by the shape of the plate sections 10, 20 and engineering and plate machining considerations understood by those skilled in the field of the disclosure. In embodiments, each of the screw holes 12, 12a and 22 are distributed within the plate sections 10, 20 such that none are located on the central axis of the ligament 30 parallel to the direction of the width 33 of the ligament 30, as shown in exemplary form in the implant device 100 of FIGS. 1A-1D. In other embodiments, a screw hole 12 and/or 22 can be located in the plate section 10 and/or 20, respectively, on or in close proximity to the central axis of the ligament 30 parallel to the direction of the width 33 of the ligament 30, as shown in exemplary form in the implant device 100 labeled “I” in FIG. 3 (described in detail below).

Still referring to FIGS. 1A-1D, the TPLO implant device 100 can be configured such that each of the tibial plate sections 10, 20 comprises a cut-adjacent curvilinear surface 16, 26 that is substantially normal to its bone-contacting surface 14b, 24b (and normal to its exterior surface 14a, 24a), the cut-adjacent curvilinear surface 16, 26 comprising a plate radius 16a, 26a within ±25% of the curvilinear cut radius 150a of the curvilinear cut 150 (see FIGS. 2A-2C, described in further detail below). In embodiments, the plate radius 16a, 26a of the cut-adjacent curvilinear surface 16, 26 of the epiphyseal and metaphyseal tibial plate sections 10, 20 are within ±25%, 20%, 15%, 10%, 5%, or even 1% of the curvilinear cut radius 150a. In embodiments, the plate radius 16a, 26a of the cut-adjacent curvilinear surface 16, 26 can range from 10 mm to 30 mm, e.g., 10 mm, 12 mm, 13 mm, 14 mm, 16 mm, 18 mm, 20 mm, 21 mm, 22 mm, 24 mm, 26 mm, 28 mm, 30 mm, or any radius value between the foregoing. According to some implementations, the plate radius 16a of the epiphyseal tibial plate section 10 and the plate radius 26a of the metaphyseal tibial plate section 20 differ by no more than +10%, +5%, or even ±1%. In general, the foregoing configurations relate to shapes of the epiphyseal and metaphyseal tibial plate sections 10, 20 with cut-adjacent curvilinear surfaces 16, 26 that substantially match the shape of the curvilinear cut 150, including its radius 150a, to ensure a uniform distribution of stress and an optimal reinforcement between the epiphyseal and metaphyseal tibial fragments 110, 120. In some embodiments, the plate radii 16a and 26a of the epiphyseal and metaphyseal tibial plate sections 10, 20, respectively, are the same or substantially similar (e.g., <±10% different from one another). In other implementations, the plate radii 16a and 26a are unequal, differing by +10%, 20%, 30%, 40%, or even 50%, and all differences between the foregoing levels (e.g., 16a=13 mm and 26a=21 mm, a difference of ~38%).

According to some embodiments of the TPLO implant device 100, each of the tibial plate sections 10, 20 comprises a respective cut-adjacent curvilinear surface 16, 26 with a shape (e.g., a concave shape) that substantially matches the shape (e.g., a concave shape) and/or alignment (e.g., a distally inward orientation) of the curvilinear cut 150 (see below). Further, according to some embodiments of the TPLO implant device 100, each of the tibial plate sections comprises a surface opposing the respective cut-adjacent curvilinear surfaces 16, 26 that is curvilinear and/or concave in shape (see FIGS. 1A-1D) that substantially matches the shape of the respective surface 16, 26. According to other implementations of the TPLO implant device 100, each, or one, of the tibial plate sections 10, 20 comprises a surface opposing the respective cut-adjacent curvilinear surface 16, 26 that is not curvilinear and/or concave in shape (e.g., as having a planar, convex, or rectilinear shape; not shown in FIGS. 1A-1D).

Referring again to the TPLO implant device 100 depicted in exemplary form in FIGS. 1A-1D, it should be understood that the curvilinear cut 150 of the tibia bone (i.e., as secured by the device 100) can be defined by a curvilinear cut radius 150a and separates the epiphyseal and metaphyseal tibial bone fragments 110, 120 (see FIGS. 2A-2C, described in further detail below). In general, the practitioner of a TPLO procedure can use an appropriate power tool, as understood by those skilled in the field of the disclosure (e.g., a deSoutter MBQ-708 Vdrive drill, as procured from Movora (a veterinary component supplier)), to make a curvilinear cut to the tibia to form a cut radius 150a that ranges from 5 mm to 35 mm. More particularly, the TPLO practitioner uses a saw blade with a sawblade number that corresponds to the desired cut radius 150a (e.g., TPLO Saw Blade Nos. 8-30, as procured from Morova (a veterinary component supplier), that correspond to cut radii 8-30 mm, respectively) to form the cut 150. For example, the curvilinear cut radius 150a of the curvilinear cut 150 can be 8 mm, 10 mm, 12 mm, 15 mm, 18 mm, 20 mm, 24 mm, 27 mm, or 30 mm. In some embodiments, the curvilinear cut 150 can be defined with a shape (e.g., a concave shape) with an alignment (e.g., a distally inward orientation) toward the metaphyseal tibial bone fragment 120.

According to another embodiment, a TPLO implant device 100 for a TPLO procedure is depicted in exemplary form in FIGS. 1A-1D. The implant device 100 according to this embodiment includes an epiphyseal tibial plate section 10 with at least two screw holes 12, an exterior surface 14a, and a bone-contacting surface 14b, each screw hole 12 of the epiphyseal tibial plate section 10 configured to accept bone screws 180 to secure the plate section 10 to an epiphyseal tibial bone fragment 110. The implant device 100 further includes a metaphyseal tibial plate section 20 with at least two screw holes 22, an exterior surface 24a, and a bone-contacting surface 24b, each screw hole 22 of the metaphyseal tibial plate section 20 configured to accept a bone screw 180 to secure the plate section 20 to a metaphyseal tibial bone fragment 120.

The implant device 100 (see FIGS. 1A-1D) of this embodiment further includes a ligament 30 configured to join the tibial plate sections 10, 20 and to span a curvilinear tibial cut 150. The curvilinear cut 150 comprises a curvilinear cut radius 150a and separates the epiphyseal and metaphyseal tibial fragments 110, 120. Each of the epiphyseal and metaphyseal tibial plate sections 10, 20 and the ligament 30 is made from an implant grade alloy. Further, the epiphyseal tibial plate section 10 comprises an epiphyseal tibial plate section length 11 and width 13, the metaphyseal tibial plate section 20 comprises a metaphyseal tibial plate section length 21 and width 23, and each tibial plate section length 11, 21 is at least two times longer than the respective tibial plate section width 13, 23. The width 13, 23 of each of the plate sections 10, 20 is substantially uniform along the length 11, 21 of each of the respective tibial plate sections 10, 20. In addition, the epiphyseal tibial plate section length 11 is shorter than the metaphyseal tibial plate section length 21.

According to another embodiment, a TPLO implant device 100 for a TPLO procedure is depicted in exemplary form in FIGS. 1A-1D. The implant device 100 according to this embodiment consists essentially of an epiphyseal tibial plate section 10, a metaphyseal tibial plate section 20, and a ligament 30 configured to join the tibial plate sections 10, 20. As such, the implant device 100 of this embodiment does not contain any additional features which could materially affect its characteristics (e.g., an additional spacer in the curvilinear cut 150, any flanges, brackets or similar features normal to the plate sections 10, 20 for positioning the device 100 within the curvilinear cut 150, and/or any appendages or protrusions extending from the plate sections 10, 20 away from the curvilinear cut 150 more than 100% of the widths 13, 23 of these plate sections). Further, in this embodiment of the implant device 100, the epiphyseal tibial plate section 10 has at least two screw holes 12 (including one elongated screw hole 12a), an exterior surface 14a, and a bone-contacting surface 14b, each screw hole 12 of the epiphyseal tibial plate section 10 configured to accept a bone screw 180 to secure the plate section 10 to an epiphyseal tibial bone fragment 110. The metaphyseal tibial plate section 20 has at least two screw holes 22, an exterior surface 24a, and a bone-contacting surface 24b, each screw hole 22 of the metaphyseal tibial plate section 20 configured to accept a bone screw 180 to secure the plate section 20 to a metaphyseal tibial bone fragment 120. In addition, the ligament 30 is configured to span a curvilinear tibial cut 150. The curvilinear cut 150 comprises a curvilinear cut radius 150a and separates the epiphyseal and metaphyseal tibial fragments 110, 120. Each of the epiphyseal and metaphyseal tibial plate sections 10, 20 and the ligament 30 is made from an implant grade alloy. Further, the epiphyseal tibial plate section 10 comprises an epiphyseal tibial plate section length 11 and width 13, the metaphyseal tibial plate section 20 comprises a metaphyseal tibial plate section length 21 and width 23, and each tibial plate section length 11, 21 is at least two times longer than the respective tibial plate section width 13, 23. The width 13, 23 of each of the plate sections 10, 20 is substantially uniform along the length 11, 21 of each of the respective tibial plate sections 10, 20.

Referring now to FIG. 1D, an end-on view of the plate sections 10, 20 of the implant device 100 of FIG. 1A is provided, as viewed along an axis parallel to the length 11, 21 of its epiphyseal and metaphyseal tibial plate sections 10, 20 and showing the screw holes 12, 22. The screw holes 12, 22 (and any elongated hole 12a, if present) run through the plate sections 10, 20 from the exterior surface 14a, 24a to the bone-contacting surface 14b, 24b. According to implementations of the implant device 100, the holes 12, 22 of the epiphyseal and metaphyseal tibial plate sections 10, 20 are round. Further, the holes 12, 12a and 22 of the plate sections 10, 20, respectively, can have countersink, beveled edges in proximity to the exterior surfaces 14a, 24a. According to some embodiments of the implant device 100, each of the holes 12, 12a, and 22 can be configured to accommodate screws 180, which are locking or non-locking in their configuration. Further, according to implementations of the implant device 100, the screw holes 12, 12a, and 22 can have a diameter that ranges from 1.5 mm to 3.5 mm (or the longest width dimension for elongated holes 12a), including any diameter value between the foregoing, and as capable of securing screws 180 with various diameters within this range. As is evident from this figure, the screw holes 22 of the metaphyseal tibial plate section 20 can be oriented in the same direction as the thickness 28 and parallel to the cut-adjacent curvilinear surface 16. In other embodiments (not shown), one or more of the screw holes 22 can be offset 5°-15° away from the curvilinear cut 150 in the distal and/or cranial directions. As is also evident from this figure, the screw holes 12 (and elongated hole 12a, if present) of the epiphyseal tibial plate section 10 can be oriented generally in the direction of the thickness 18 and generally parallel to the cut-adjacent curvilinear surface 26, but with an offset of about 5-15° away from the curvilinear cut 150 in the caudal and/or proximal directions (see FIGS. 2A-2C). For example, the screw holes 12 (and elongated hole 12a, if present) can be offset 5°, 10°, 15°, or all angles between the foregoing, away from the curvilinear cut 150 in the caudal and/or proximal directions (see FIGS. 2A-2C). In other embodiments, one or more of the screw holes 12 (and elongated hole 12a, if present) can be oriented generally in the direction of the thickness 18 and parallel to the cut-adjacent curvilinear surface 26.

Referring now to FIGS. 2A-2C, an implant assembly 200 is shown in various perspective views. The implant assembly 200 includes a TPLO implant device 100 configured according to principles of the disclosure (see FIGS. 1A-1D and corresponding description above), as secured to epiphyseal and metaphyseal tibial bone fragments 110, 120 across a curvilinear tibial cut 150. As is evident from these figures, the implant device 100 is installed by a practitioner during a TPLO procedure. Notably, the practitioner installs the implant device 100 after the curvilinear cut 150 is made to separate the epiphyseal and metaphyseal tibial bone fragments 110, 120 (e.g., as using Saw Blade Nos. 8-30 from Movora), and the epiphyseal tibial bone fragment 110 has been rotated in a clockwise direction for the left leg and in a counterclockwise direction for the right leg along the curvilinear cut 150 to level the tibial bone plateau 110a. Once the tibial bone plateau 110a has been leveled by the practitioner, the implant device 100 is affixed to the epiphyseal and metaphyseal tibial bone fragments 110, 120 by bone screws 180, as inserted through holes 12, 22, and any elongated hole 12a, if present. Unless otherwise noted, bone screws 180 are bone screws suitable for affixing implant grade alloy plate(s), such as used to fabricate implant device 100, to tibial bone fragments, as understood by those skilled in the field of the disclosure. In some embodiments, the bone screws 180 have one threaded region in which the threads are configured to interface with the tibial bone fragments. In other embodiments, the bone screws 180 have two threaded regions, with a first region in proximity to the head of the screw 180 for interfacing with threads of the screw holes 12, 22 and 12a, if present, and the second threaded region for interfacing with the underlying tibial bone fragments (not shown). For embodiments of the implant device 100 (see FIGS. 2A-2C) that comprise an elongated hole 12a in the epiphyseal tibial plate section 10, the screw 180 used in this hole is adjusted by the practitioner within the hole relative to the curvilinear cut 150 to increase the compressive forces between the tibial bone fragments 110, 120. As is also evident from the figures, the implant device 100 is installed such that ligament 30 spans the curvilinear cut 150 and the cut-adjacent curvilinear surfaces 16, 26 of the epiphyseal and metaphyseal tibial plate sections 10, 20 are aligned along the curvilinear cut 150.

According to an embodiment of the implant assembly 200, as depicted in FIGS. 2A-2C, a practitioner can employ an installation method that maximizes and builds uniform compressive forces between the epiphyseal and metaphyseal tibial bone fragments 110, 120. In this embodiment, the practitioner first secures the metaphyseal tibial plate section 20 to the metaphyseal tibial bone fragment 120, placing one bone screw 180 into one of the screw holes 12 in the epiphyseal tibial plate section 10 located proximally to the ligament 30 and one of the screw holes 22 in the metaphyseal tibial plate section 20 located distally to the ligament 30. Next, the practitioner secures the epiphyseal tibial plate section 10 to the epiphyseal tibial bone fragment 110 by placing a bone screw 180 into one of the most proximal (closest to the curvilinear cut 150) screw holes 12 in a compressing position. Finally, the practitioner further secures the implant device 100 to the two tibial bone fragments 110, 120 by inserting bone screws 180 into the remaining screw holes 12, 22 in the plate sections 10, 20, respectively.

Referring now to FIG. 3, a top plan view is provided of nine (9) TPLO implant device 100 embodiments with epiphyseal and metaphyseal tibial plate sections 10, 20 having varying sizes and bone screw hole 12, 12a, and 22 configurations for animals of various sizes (labeled “A” through “I”). As is evident from the figure, implant devices 100 with particular screw hole 12, 12a, and 22 and epiphyseal and metaphyseal plate section length 11, 21 (see FIGS. 1A-1D) configurations set forth below in Table 1 are depicted. It should be recognized that the implant device 100 configurations in FIG. 3 and Table 1 below are merely illustrative. In addition, it should be recognized that the implant device 100 configurations shown in FIG. 3 are not necessarily to scale. In general, the implant devices 100 labeled “A” through “H” in FIG. 3 successively increase in size and are indicative of implant devices that can be selected by a practitioner to accommodate canines with different sizes. Other configurations of the implant device 100 are also consistent with the principles outlined earlier in this disclosure (e.g., an epiphyseal tibial plate section 10 with three (3) screw holes 12 and a metaphyseal tibial plate section 20 with five (5) screw holes 22, an epiphyseal tibial plate section 10 with four (4) screw holes 12 and a metaphyseal tibial plate section 20 with four (4) screw holes 22, etc.).

TABLE 1
Illustrative TPLO implant device configurations

| | # of | | # of | |
|---|---|---|---|---|
| | epiphyseal | epiphyseal | metaphyseal | metaphyseal |
| TPLO implant | tibial plate | tibial plate | tibial plate | tibial plate |
| device | section screw | section | section screw | section |
| (See FIG. 3) | holes | length (mm) | holes | length (mm) |
| A | 2 | 8 | 4 | 10 |
| B | 2 | 13 | 4 | 15 |
| C | 4 | 17 | 4 | 19 |
| D | 4 | 19 | 4 | 21 |
| E | 4 | 20 | 4 | 22 |
| F | 4 | 25 | 6 | 27 |
| G | 6 | 26 | 6 | 28 |
| H | 6 | 30 | 6 | 32 |
| I | 3 | 20 | 4 | 25 |
| I | 3 | 24 | 4 | 32 |

Referring again to FIG. 3, the implant device 100 labeled “I” is an exemplary implant device 100 embodiment with three (3) screw holes 12 in its epiphyseal tibial plate section 10 and four (4) screw holes 22 in its metaphyseal tibial plate section 20. Further, one of the screw holes 12 is located on the central axis of the ligament 30 parallel to the direction of its width (e.g., width 33, as described earlier in the disclosure). Further, as noted in Table 1, exemplary dimensions of the implant device 100 labeled “I” in FIG. 3 can include an epiphyseal tibial plate section length 11 of 20-24 mm and a metaphyseal tibial plate section length 21 of 25-32 mm. Other exemplary dimensions of the implant device 100 labeled “I” in FIG. 3 include epiphyseal and metaphyseal tibial plate section widths 13, 23 of 6-8 mm, ligament length 31 of 6-8 mm, ligament width 33 of 6-8 mm, and a plate radius 26a of the cut-adjacent curvilinear surface 26 of the metaphyseal tibial plate section 21 of about 21 mm.

Referring now to FIG. 4, a top plan view is provided of a TPLO implant device 100 with epiphyseal and metaphyseal tibial plate sections 10, 20 and a ligament 30. Notably, each plate section 10, 20 of the implant device 100 shown in FIG. 4 can include one or more short, support protrusions 17, 27, as shown in exemplary form. As mentioned earlier, in some embodiments of the implant device 100 (see also FIGS. 1A-1D), one or both of the plate sections 10, 20 can have one or more short, support protrusions 17, 27. In some implementations, each of the short, support protrusions 17, 27 comprises a screw hole 12, 22, respectively, and emanates from a surface of the plate sections 10, 20 opposite to the cut-adjacent curvilinear surface 16, 26. In general, these support protrusion(s) 17, 27 are limited in width 17a, 27a to no more than 100% of the width 13, 23 (see FIG. 1B) of the plate sections 10, 20. For example, the support protrusion(s) 17, 27 can have a width 17a, 27a, respectively, of 100%, 95%, 90%, 85%, 80%, 75%, 70%, 65%, 60%, 55%, 50%, 45%, 40%, 35%, 30%, or even 25% of the width 13, 23 (see FIG. 1B) of the plate sections 10, 20, respectively. As such, the support protrusion(s) 17 extends only to a limited degree along the epiphyseal tibial bone fragment 110 toward the tibial plateau 110a (see FIGS. 2A-2C). Similarly, the support protrusion(s) 27 extends only to a limited degree away from the curvilinear cut 150 along the metaphyseal tibial bone fragment 120. Consequently, even if one or more short, support protrusion(s) 17, 27 are present in the implant device 100 according to this embodiment, the implant device 100 remains compact and only a small incision site is needed as part of the TPLO procedure.

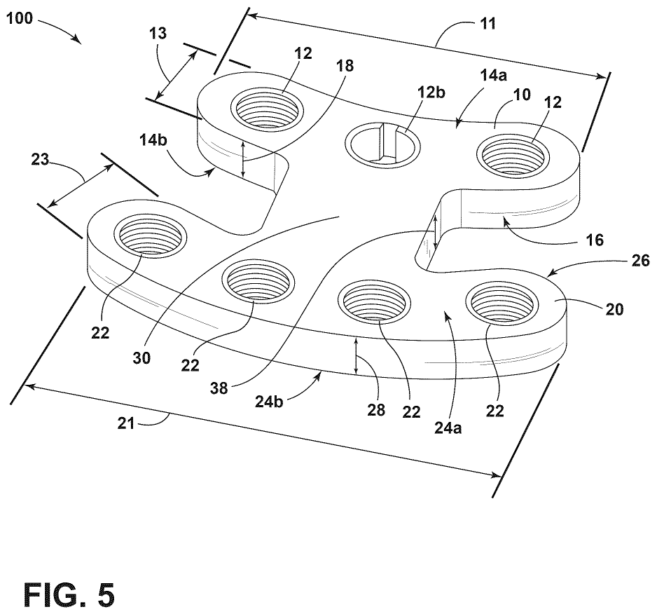

Referring now to FIG. 5, a perspective view is provided of a TPLO implant device 100 with epiphyseal and metaphyseal tibial plate sections 10, 20 and a ligament 30. Unless otherwise noted, the implant device 100 of FIG. 5 has the same features with the same functions as the implant device 100 shown in FIGS. 1A-1D, as described above, with only a few exceptions. Notably, the plate section 10 of this implant device 100 embodiment can be configured with two to eight screw holes 12, with one screw hole 12 located proximal to the ligament 30, or on or in proximity to the central axis of the ligament 30 that runs parallel to its width 33 (see FIG. 1). As shown in exemplary form in FIG. 5, the plate section 10 has three holes 12. Further, the plate section 20 of this implant device 100 embodiment can be configured with two to eight screw holes 22, with four holes 22 in the plate section 20 as depicted in exemplary form in FIG. 5.

Referring again to the embodiment of the implant device 100 depicted in FIG. 5, each of the screw holes 12, 22 of the plate sections 10, 20 are configured with a beveled region adjacent to the exterior surfaces 14a, 24a. In some embodiments, some or all the holes 12, 22 in the plate sections 10, 20 can possess a threaded region that runs through the thicknesses 18, 28 of the plate sections 10, 20 to the opposite bone-contacting surfaces 14b, 24b. Accordingly, bone screws 180 (not shown) with a tapered head (e.g., that fits the bevel of the screw holes 12, 22) can be threaded into the screw holes 12, 22 and into the underlying tibial bone fragments to secure the implant device 100 to the tibial bone fragments. In some embodiments, bone screws 180 (not shown) with two threaded regions can be employed with the implant device 100 embodiment of FIG. 5, the first threaded region toward the head of the screws 180 configured for threading into the threads of the screw holes 12, 22 and the second threaded region toward the tip of the screws 180 configured for drilling into the underlying tibial bone fragment.

Further, in some implementations of this embodiment of the implant device 100, as depicted in exemplary form in FIG. 5, the centermost screw hole 12b in the epiphyseal tibial plate section 10 can be configured without threads and with an unbeveled region without any bevel, chamfer or the like in its uppermost portion in proximity to the exterior surface 14a. The region of the screw hole 12b without or devoid of beveling can range from 315° to 45° (90° in total), 330° to 30° (60° in total), 345° to 15° (45° in total), or any range within the foregoing endpoints of the total circumference of the screw hole 12b, such that 0° is positioned at the uppermost edge of the screw hole 12b opposite from the ligament 30 (and, also, opposite from the curvilinear cut 150), as shown in FIG. 5. According to an embodiment, a practitioner can use a bone screw 180 with a beveled head (not shown, e.g., with one or two threaded regions), position it toward the region without beveling of the centermost screw hole 12b, and then drill the screw 180 into the underlying tibial bone fragment such that the head of the screw 180 substantially compresses the upper portion of the screw hole 12b with and without beveling. It is believed that these embodiments of the implant device 100 with a centermost screw hole 12b in the epiphyseal tibial plate section 10 without threading and a region without beveling, as shown in FIG. 5, have the benefit of being able to be secured with increased compressive force between the underlying epiphyseal tibial bone fragment(s) 110 and the metaphyseal tibial plate section 20. Furthermore, it is believed that the region of the screw hole 12b without beveling enables the screw 180 (not shown) to produce the most compressive force between the epiphyseal and metaphyseal tibial bone fragments 110, 120 because more displacement can occur in this region as the screw 180 is inserted in the hole 12b and into the epiphyseal tibial bone fragment 110.

Aspect 1. An implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament that joins the tibial plate sections and is configured to span a curvilinear tibial cut, wherein the curvilinear cut separates the epiphyseal and metaphyseal tibial bone fragments and has a concave shape with a distally inward orientation toward the metaphyseal tibial bone fragment. Each of the metaphyseal and epiphyseal tibial plate sections and the ligament comprises an implant grade alloy, the epiphyseal tibial plate section comprises an epiphyseal tibial plate length and width, the metaphyseal tibial plate comprises a metaphyseal tibial plate length and width, and each tibial plate section length is at least two times longer than the respective tibial plate width. Each of the tibial plate sections comprises a cut-adjacent curvilinear surface that is substantially normal to its bone-contacting surface. Each of the cut-adjacent curvilinear surfaces has a concave shape that substantially matches the concave shape and inward orientation of the curvilinear cut.

Aspect 2. The implant device of Aspect 1, wherein each of the tibial plate sections comprises two (2) to eight (8) screw holes and a curvilinear shape.

Aspect 3. The implant device of Aspect 1, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

Aspect 4. The implant device of Aspect 3, wherein the ligament further comprises a ligament width from 4 mm to 10 mm and a ligament length from 4 mm to 12 mm, and further wherein each of the plate sections and the ligament comprises a thickness from 1.5 mm to 4 mm.

Aspect 5. The implant device of Aspect 1, wherein the width of each of the tibial plate sections along the length of each of the respective tibial plate sections is both uniform and varies by no more than ±25%.

Aspect 6. The implant device of Aspect 1, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 7. The implant device of Aspect 1, wherein one of the at least two screw holes of the epiphyseal plate section is elongated in a direction perpendicular to the curvilinear tibial cut.

Aspect 8. An implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament that joins the tibial plate sections and is configured to span a curvilinear tibial cut, wherein the curvilinear cut separates the epiphyseal and metaphyseal tibial fragments and has a shape with a distally inward orientation toward the metaphyseal tibial bone fragment. Each of the epiphyseal and metaphyseal tibial plate sections and the ligament comprises an implant grade alloy. The epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate section width. The epiphyseal tibial plate section length is shorter than the metaphyseal tibial plate section length. Each of the tibial plate sections comprises a cut-adjacent curvilinear surface that is substantially normal to its bone-contacting surface. Further, each of the cut-adjacent curvilinear surfaces has a shape that substantially matches the shape and inward orientation of the curvilinear cut.

Aspect 9. The implant device of Aspect 8, wherein each of the tibial plate sections comprises two (2) to eight (8) screw holes and a curvilinear shape.

Aspect 10. The implant device of Aspect 8, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

Aspect 11. The implant device of Aspect 10, wherein the ligament further comprises a ligament width from 4 mm to 10 mm and a ligament length from 4 mm to 12 mm, and further wherein each of the plate sections and the ligament comprises a thickness from 1.5 mm to 4 mm.

Aspect 12. The implant device of Aspect 8, wherein the width of each of the tibial plate sections along the length of each of the respective tibial plate sections is both uniform and varies by no more than +25%.

Aspect 13. The implant device of Aspect 8, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 14. The implant device of Aspect 8, wherein one of the at least two screw holes of the epiphyseal plate section is elongated in a direction perpendicular to the curvilinear tibial cut.

Aspect 15. An implant device for a tibial plateau leveling osteotomy is provided that consists essentially of: an epiphyseal tibial plate section comprising at least two screw holes and a bone-contacting surface, each screw hole of the epiphyseal tibial plate configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament that joins the tibial plate sections and is configured to span a curvilinear tibial cut, wherein the curvilinear cut separates the epiphyseal and metaphyseal tibial fragments and has a shape with a distally inward orientation toward the metaphyseal tibial bone fragment. Each of the epiphyseal and metaphyseal tibial plate sections and the ligament comprises an implant grade alloy. The epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate section width. In addition, at least one screw hole of the epiphyseal tibial plate section is devoid of threading and comprises an unbeveled region, the unbeveled region comprising 90° or less of the total circumference of the at least one screw hole. Each of the tibial plate sections comprises a cut-adjacent curvilinear surface that is substantially normal to its bone-contacting surface. Further, each of the cut-adjacent curvilinear surfaces has a shape that substantially matches the shape and inward orientation of the curvilinear cut.

Aspect 16. The implant device of Aspect 15, wherein each of the tibial plate sections comprises two (2) to eight (8) screw holes and a curvilinear shape.

Aspect 17. The implant device of Aspect 15, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

Aspect 18. The implant device of Aspect 15, wherein the width of each of the tibial plate sections along the length of each of the respective tibial plate sections is both uniform and varies by no more than ±25%.

Aspect 19. The implant device of Aspect 15, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 20. The implant device of Aspect 15, wherein the shape of the curvilinear cut and the cut-adjacent curvilinear surfaces is concave.

Aspect 21. An implant device for a tibial plateau leveling osteotomy is provided that includes: an epiphyseal tibial plate section comprising at least two screw holes and a bone-contacting surface, each screw hole of the epiphyseal tibial plate section configured to secure a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to an metaphyseal tibial bone fragment; and a ligament configured to join the tibial plates and span a curvilinear tibial cut, wherein the curvilinear cut comprises a curvilinear cut radius and separates the epiphyseal and metaphyseal tibial bone fragments. Each of the metaphyseal and epiphyseal tibial plate sections and the ligament comprises an implant grade alloy. The epiphyseal tibial plate section comprises an epiphyseal tibial plate length and width, the metaphyseal tibial plate section comprises a metaphyseal tibial plate length and width, and each tibial plate length is at least two times longer than the respective tibial plate width. The width of each of the tibial plate sections is substantially uniform along the length of each of the respective tibial plate sections. Further, each of the tibial plate sections comprises a cut-adjacent curvilinear surface that is substantially normal to its bone-contacting surface, the curvilinear surface comprising a plate radius within +25% of the curvilinear cut radius.

Aspect 22. The implant device of Aspect 21, wherein the cut-adjacent curvilinear surface of each plate comprises a plate radius within 10% of the cut radius.

Aspect 23. The implant device of Aspect 21, wherein each of the tibial plate sections comprises two (2) to eight (8) screw holes and a curvilinear shape.

Aspect 24. The implant device of Aspect 21, wherein the ligament comprises a ligament length defined along an axis substantially parallel to an axis defined by each of the epiphyseal and metaphyseal tibial plate lengths, the ligament length is at least 50% shorter than each of the epiphyseal and metaphyseal tibial plate lengths.

Aspect 25. The implant device of Aspect 24, wherein the ligament further comprises a ligament width from 4 mm to 10 mm and a ligament length from 4 mm to 12 mm, and further wherein each of the plate sections and the ligament comprises a thickness from 1.5 mm to 4 mm.

Aspect 26. The implant device of Aspect 21, wherein the width of each of the tibial plate sections along the length of each of the respective tibial plate sections is both uniform and varies by no more than ±5%.

Aspect 27. The implant device of Aspect 21, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the epiphyseal tibial plate section width is from 4 mm to 10 mm.

Aspect 28. The implant device of Aspect 21, wherein the plate radius of each of the curvilinear surfaces of the epiphyseal and metaphyseal tibial plate section is from 10 mm to 30 mm, and further wherein the plate radii of the curvilinear surfaces of the tibial plate sections are within 5% of one another.

Aspect 29. The implant device of Aspect 21, wherein one of the at least two screw holes of the epiphyseal plate section is elongated in a direction perpendicular to the curvilinear tibial cut.

Aspect 30. An implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament configured to join the tibial plate sections and to span a curvilinear tibial cut, wherein the curvilinear cut comprises a curvilinear cut radius and separates the epiphyseal and metaphyseal tibial fragments. Each of the epiphyseal and metaphyseal tibial plate sections and the ligament comprises an implant grade alloy. Further, the epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate section width. The width of each of the plate sections is substantially uniform along the length of each of the respective tibial plate sections. In addition, the epiphyseal tibial plate section length is shorter than the metaphyseal tibial plate section length.

Aspect 31. The implant device of Aspect 30, wherein each of the tibial plate sections comprises two (2) to eight (8) screw holes and a curvilinear shape.

Aspect 32. The implant device of Aspect 30, wherein the ligament comprises a ligament length defined along an axis substantially parallel to an axis defined by each of the epiphyseal and metaphyseal tibial plate section lengths, the ligament length is at least 50% shorter than each of the epiphyseal and metaphyseal tibial plate section lengths.

Aspect 33. The implant device of Aspect 32, wherein the ligament further comprises a ligament width from 4 mm to 10 mm and a ligament length from 4 mm to 12 mm, and further wherein each of the plate sections and the ligament comprises a thickness from 1.5 mm to 4 mm.

Aspect 34. The implant device of Aspect 30, wherein the width of each of the tibial plate sections along the length of each of the respective tibial plate sections is both uniform and varies by no more than ±5%.

Aspect 35. The implant device of Aspect 30, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 36. The implant device of Aspect 30, wherein each of the tibial plate sections comprises a cut-adjacent curvilinear surface that is substantially normal to its bone-contacting surface, the curvilinear surface comprising a plate radius within +25% of the curvilinear cut radius.

Aspect 37. The implant device of Aspect 30, wherein one of the at least two screw holes of the epiphyseal plate section is elongated in a direction perpendicular to the curvilinear tibial cut.

Aspect 38. An implant device for a tibial plateau leveling osteotomy is provided that consists essentially of: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament configured to join the tibial plates and to span a curvilinear tibial cut, wherein the curvilinear cut comprises a curvilinear cut radius and is separates the epiphyseal and metaphyseal tibial fragments. Each of the epiphyseal and metaphyseal tibial plate sections and the ligament comprises an implant grade alloy. Further, the epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate width. In addition, the width of each of the plate sections is substantially uniform along the length of each of the respective tibial plate sections.

Aspect 39. The implant device of Aspect 38, wherein each of the tibial plate sections comprises two (2) to eight (8) screw holes and a curvilinear shape.

Aspect 40. The implant device of Aspect 38, wherein the ligament comprises a ligament length defined along an axis substantially parallel to an axis defined by each of the epiphyseal and metaphyseal tibial plate section lengths, the ligament length is at least 50% shorter than each of the epiphyseal and metaphyseal tibial plate section lengths, and further wherein the ligament comprises a ligament width, the ligament width is from 4 mm to 10 mm, and the ligament length is from 4 mm to 12 mm.

Aspect 41. The implant device of Aspect 38, wherein each of the plate sections and the ligament comprises a thickness from 1.5 mm to 4 mm, and further wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 42. The implant device of Aspect 38, wherein each of the tibial plate sections comprises a cut-adjacent curvilinear surface that is substantially normal to its bone-contacting surface, the curvilinear surface comprising a plate radius within +25% of the curvilinear cut radius, and further wherein the epiphyseal tibial plate section length is shorter than the metaphyseal tibial plate length.

Aspect 43. The implant device of Aspect 38, wherein one of the at least two screw holes of the epiphyseal plate section is elongated in a direction perpendicular to the curvilinear tibial cut.

Aspect 44. An implant device for a tibial plateau leveling osteotomy is provided that includes: an epiphyseal tibial plate section comprising at least two screw holes and a bone-contacting surface, each screw hole of the epiphyseal tibial plate section configured to accept a bone screw to secure the plate section to an epiphyseal tibial bone fragment; a metaphyseal tibial plate section comprising at least two screw holes and a bone-contacting surface, each screw hole of the metaphyseal tibial plate section configured to accept a bone screw to secure the plate section to a metaphyseal tibial bone fragment; and a ligament configured to join the tibial plates and to span a curvilinear tibial cut, wherein the curvilinear cut comprises a curvilinear cut radius and is separates the epiphyseal and metaphyseal tibial fragments. Each of the epiphyseal and metaphyseal tibial plate sections and the ligament comprises an implant grade alloy. Further, the epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate width. In addition, the width of each of the plate sections is substantially uniform along the length of each of the respective tibial plate sections. Further, one or both of the tibial plate sections comprises a support protrusion, the support protrusion comprising a screw hole and emanating from a surface of the plate section opposite to a cut-adjacent curvilinear surface of the plate section that is substantially normal to its bone-contacting surface.

Aspect 45. The implant device of Aspect 44, wherein each support protrusion comprises a width that is no more than 100% of the width of the respective plate section.

Aspect 46. The implant device of Aspect 44, wherein each of the tibial plate sections comprises two (2) to eight (8) screw holes and a curvilinear shape.

Aspect 47. The implant device of Aspect 44, wherein the ligament comprises a ligament length defined along an axis substantially parallel to an axis defined by each of the epiphyseal and metaphyseal tibial plate section lengths, the ligament length is at least 50% shorter than each of the epiphyseal and metaphyseal tibial plate section lengths, and further wherein the ligament comprises a ligament width, the ligament width is from 4 mm to 10 mm, and the ligament length is from 4 mm to 12 mm.

Aspect 48. The implant device of Aspect 44, wherein each of the plate sections and the ligament comprises a thickness from 1.5 mm to 4 mm, and further wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

Aspect 49. The implant device of Aspect 44, wherein each of the tibial plate sections comprises a cut-adjacent curvilinear surface that is substantially normal to its bone-contacting surface, the curvilinear surface comprising a plate radius within +25% of the curvilinear cut radius, and further wherein the epiphyseal tibial plate section length is shorter than the metaphyseal tibial plate length.

Aspect 50. The implant device of Aspect 44, wherein one of the at least two screw holes of the epiphyseal plate section is elongated in a direction perpendicular to the curvilinear tibial cut.

Although multiple embodiments of the TPLO implant device of the present disclosure have been illustrated in the accompanying Drawings and described in the foregoing Detailed Description, it should be understood that the present disclosure is not limited to the disclosed embodiments. Rather, the osteotomy implant device is also capable of numerous rearrangements (e.g., implant devices with epiphyseal and metaphyseal tibial plate sections having multiple sections, some or all in a curvilinear shape; implant devices with ligaments formed in a mesh to join the tibial plate sections while retaining visibility to the curvilinear cut below, etc.), modifications, and/or substitutions without departing from the present disclosure that has been set forth and defined within the following claims.

What is claimed is:

1. An implant device for a tibial plateau leveling osteotomy, comprising:
an epiphyseal tibial plate section comprising at least two screw holes and a bone-contacting surface, each screw hole of the epiphyseal tibial plate section configured to accept a bone screw to secure the plate section to an epiphyseal tibial bone fragment;
a metaphyseal tibial plate section comprising at least two screw holes and a bone-contacting surface, each screw hole of the metaphyseal tibial plate section configured to accept a bone screw to secure the plate section to a metaphyseal tibial bone fragment; and
a ligament that joins the tibial plate sections and is configured to span a curvilinear tibial cut, wherein the curvilinear cut separates the epiphyseal and metaphyseal tibial bone fragments and has a concave shape with a distally inward orientation toward the metaphyseal tibial bone fragment,
wherein each of the metaphyseal and epiphyseal tibial plate sections and the ligament comprises an implant grade alloy,
wherein the epiphyseal tibial plate section comprises an epiphyseal tibial plate length and width, the metaphyseal tibial plate comprises a metaphyseal tibial plate length and width, and each tibial plate section length is at least two times longer than the respective tibial plate width,
wherein each of the tibial plate sections comprises a cut-adjacent curvilinear surface that is substantially normal to its bone-contacting surface, and
further wherein each of the cut-adjacent curvilinear surfaces has a concave shape that substantially matches the concave shape and inward orientation of the curvilinear cut.

2. The implant device of claim 1, wherein each of the tibial plate sections comprises two (2) to eight (8) screw holes and a curvilinear shape.

3. The implant device of claim 1, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

4. The implant device of claim 3, wherein the ligament further comprises a ligament width from 4 mm to 10 mm and a ligament length from 4 mm to 12 mm, and further wherein each of the plate sections and the ligament comprises a thickness from 1.5 mm to 4 mm.

5. The implant device of claim 1, wherein the width of each of the tibial plate sections along the length of each of the respective tibial plate sections is both uniform and varies by no more than 25%.

6. The implant device of claim 1, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

7. The implant device of claim 1, wherein one of the at least two screw holes of the epiphyseal plate section is elongated in a direction perpendicular to the curvilinear tibial cut.

8. An implant device for a tibial plateau leveling osteotomy, comprising:
an epiphyseal tibial plate section comprising at least two screw holes and a bone-contacting surface, each screw hole of the epiphyseal tibial plate section configured to accept a bone screw to secure the plate section to an epiphyseal tibial bone fragment;
a metaphyseal tibial plate section comprising at least two screw holes and a bone-contacting surface, each screw hole of the metaphyseal tibial plate section configured to accept a bone screw to secure the plate section to a metaphyseal tibial bone fragment; and
a ligament that joins the tibial plate sections and is configured to span a curvilinear tibial cut, wherein the curvilinear cut separates the epiphyseal and metaphyseal tibial fragments and has a shape with a distally inward orientation toward the metaphyseal tibial bone fragment,
wherein each of the epiphyseal and metaphyseal tibial plate sections and the ligament comprises an implant grade alloy,
wherein the epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate section width,
wherein the epiphyseal tibial plate section length is shorter than the metaphyseal tibial plate section length,
wherein each of the tibial plate sections comprises a cut-adjacent curvilinear surface that is substantially normal to its bone-contacting surface, and
further wherein each of the cut-adjacent curvilinear surfaces has a shape that substantially matches the shape and inward orientation of the curvilinear cut.

9. The implant device of claim 8, wherein each of the tibial plate sections comprises two (2) to eight (8) screw holes and a curvilinear shape.

10. The implant device of claim 8, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

11. The implant device of claim 10, wherein the ligament further comprises a ligament width from 4 mm to 10 mm and a ligament length from 4 mm to 12 mm, and further wherein each of the plate sections and the ligament comprises a thickness from 1.5 mm to 4 mm.

12. The implant device of claim 8, wherein the width of each of the tibial plate sections along the length of each of the respective tibial plate sections is both uniform and varies by no more than 25%.

13. The implant device of claim 8, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

14. The implant device of claim 8, wherein one of the at least two screw holes of the epiphyseal plate section is elongated in a direction perpendicular to the curvilinear tibial cut.

15. An implant device for a tibial plateau leveling osteotomy, consisting essentially of:
an epiphyseal tibial plate section comprising at least two screw holes and a bone-contacting surface, each screw hole of the epiphyseal tibial plate section configured to accept a bone screw to secure the plate section to an epiphyseal tibial bone fragment;
a metaphyseal tibial plate section comprising at least two screw holes and a bone-contacting surface, each screw hole of the metaphyseal tibial plate section configured to accept a bone screw to secure the plate section to a metaphyseal tibial bone fragment; and
a ligament that joins the tibial plate sections and is configured to span a curvilinear tibial cut, wherein the curvilinear cut separates the epiphyseal and metaphyseal tibial fragments and has a shape with a distally inward orientation toward the metaphyseal tibial bone fragment,
wherein each of the epiphyseal and metaphyseal tibial plate sections and the ligament comprises an implant grade alloy,
wherein the epiphyseal tibial plate section comprises an epiphyseal tibial plate section length and width, the metaphyseal tibial plate section comprises a metaphyseal tibial plate section length and width, and each tibial plate section length is at least two times longer than the respective tibial plate section width,
wherein at least one screw hole of the epiphyseal tibial plate section is devoid of threading and comprises an unbeveled region, the unbeveled region comprising 90° or less of the total circumference of the at least one screw hole,
wherein each of the tibial plate sections comprises a cut-adjacent curvilinear surface that is substantially normal to its bone-contacting surface, and
further wherein each of the cut-adjacent curvilinear surfaces has a shape that substantially matches the shape and inward orientation of the curvilinear cut.

16. The implant device of claim 15, wherein each of the tibial plate sections comprises two (2) to eight (8) screw holes and a curvilinear shape.

17. The implant device of claim 15, wherein the ligament comprises a ligament length defined along an axis substantially parallel to an axis defined by each of the epiphyseal and metaphyseal tibial plate lengths, the ligament length is at least 35% shorter than each of the epiphyseal and metaphyseal tibial plate lengths.

18. The implant device of claim 15, wherein the width of each of the tibial plate sections along the length of each of the respective tibial plate sections is both uniform and varies by no more than ±25%.

19. The implant device of claim 15, wherein the epiphyseal tibial plate section length is from 8 mm to 36 mm, the epiphyseal tibial plate section width is from 4 mm to 10 mm, the metaphyseal tibial plate section length is from 10 mm to 38 mm, and the metaphyseal tibial plate section width is from 4 mm to 10 mm.

20. The implant device of claim 15, wherein the shape of the curvilinear cut and the cut-adjacent curvilinear surfaces is concave.
